(19) United States
(12) Patent Application Publication
(10) Pub. No.: US 2008/0146941 A1
(43) Pub. Date: Jun. 19, 2008

(54) Catheter Position Tracking for Intracardiac Catheters

(75) Inventor: Praveen Dala-Krishna, Sicklerville, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MEDSYSTEMS, INC., West Berlin, NJ (US)

(21) Appl. No.: 11/610,357

(22) Filed: Dec. 13, 2006

(51) Int. Cl. A61B 8/00

(52) U.S. Cl. 600/466

(57) ABSTRACT

An ultrasound imaging system includes one or more accelerometers positioned near the imaging transducer. Acceleration data from the accelerometers are used to estimate the position of the imaging transducer. By combining position information calculated based on acceleration data with position information obtained by other methods, the imaging transducer position can be determined more accurately and closer in time to when images are obtained. The resulting accurate imaging transducer position information enables combining multiple images from different positions or orientations to generate multi-dimensional images.

### BACKGROUND OF THE INVENTION

1. Field of the Invention

The present invention relates generally to tracking the position of catheters used in medical procedures that are introduced into the human body, and more particularly to a method and apparatus for tracking ultrasound imaging catheters to ascertain image plane orientation and position.

2. Description of the Related Art

Ultrasound devices have been developed and refined for the diagnosis and treatment of various medical conditions. Such devices have been developed, for example, to track the magnitude and direction of motion of moving objects, and/or the position of moving objects over time. By way of example, Doppler echocardiography is one ultrasound technique used to determine motion information from the recording and measurement of Doppler data for the diagnosis and treatment of cardiac conditions, and is described in U.S. Patent Application Publication No. 20040127798 of U.S. application Ser. No. 10/620,517 to Dala-Krishna, the entire contents of both of which are incorporated herein by reference.

Another ultrasound imaging technique is a class generally referred to as brightness mode (“B-Mode”) display. To generate a B-Mode display, the delay and amplitude of the received energy of ultrasound pulse echoes along different coplanar lines are measured. In B-Mode echocardiography, ultrasound energy is transmitted and subsequently reflected from the endocardial surface as well as from tissue layers within the heart. Reflected ultrasound is detected by a phased array ultrasound transducer, where the sound energy is converted into electrical pulses which can be processed to determine the direction or line from which each echo is received. The received signal amplitude along a line-of-sight is used to modulate the brightness of a line of pixels corresponding to the length of the received line, determined based upon the delay in time of the received echo, and the spatial orientation of the line. A display is thus rendered from a collection of ultrasound data where the position of each “dot” corresponds to the distance from the ultrasound transducer of a given sound reflecting object, and the brightness of each “dot” corresponds to the signal strength received from that position. A collection of coplanar lines thus forms a cross-sectional image of the subject under investigation.

The current state of the art includes ultrasonic transducers deployed in various configurations at the tips of catheters that can be introduced into the circulatory system to image various parts of the body, particularly the heart and the vascular system. Linear phased arrays, circular phased arrays, and single crystal mechanically scanning transducers are commercially available. An example of an intracardiac linear phased array ultrasound transducer is the ViewMate™ which is commercially available from EP MedSystems, Inc. of West Berlin, N.J.

The aforementioned intravascular and intracardiac ultrasound imaging techniques, combined with various imaging modalities that are possible in some of these configurations (such as Doppler and Color Doppler) have given clinicians a wide variety of tools with which to diagnose and treat various medical conditions. These tools are limited, however, in their ability to provide a comprehensive view of the underlying anatomy, and their ability to accurately track and display a plurality of moving structures and instruments, such as valves and catheters, that might be required for diagnostic or treatment purposes. 2-dimensional (2-D) images provided by known ultrasound imaging systems limit viewing to the tomographic plane currently being imaged and do not provide optimal views of structures or instruments that are not coplanar to the tomographic plane. The physician is often required to continually move the imaging catheter to “trace” a structure of interest that traverses the imaging plane. Thus, a need exists for an improved method of processing ultrasound images that allows 3-D (3-D) reconstruction of the field of view.

Although 3-D reconstruction of general ultrasonic and echocardiography images is common in the field of conventional (i.e., non-catheter) ultrasound imaging, the reconstruction of 3-D ultrasonic images using catheter based transducers has proven to be a technological challenge. Such reconstruction not only requires the ability to track and acquire images in synchrony with the cardiac activity of the subject under investigation since the heart is constantly in motion (this is termed 4-D to account for the three physical dimensions and the fourth time-dimension), but one has to accurately measure and record the relative position and orientation of the imaging plane at each viewing instant. Current catheter tracking systems common in the art, such as the use of ultrasonic ranging, use of electromagnetic fields, or body electrical impedance techniques, can be quite complicated to engineer. In particular, challenges exist in determining the rotational position of a side-firing phased array catheter with sufficient accuracy to enable reconstructing a clinically useful 3-D image.

Other methods of mechanically controlling the motion of the catheter have also been attempted. Such techniques limit the ability of the physician to manipulate the catheter while adding complexity and risk to the overall patient safety and efficacy situation. 2-D arrays capable of real-time 3-D intracardiac echocardiography have been reported in literature. However, given the severe size limitations of catheters and associated element limitations, optimal image quality has not been achieved using such techniques. Thus a need exists for a simple position tracking system that provides sufficient orientation accuracy to enable 3-D reconstruction and an associated 3-D reconstruction methodology.

### SUMMARY OF THE INVENTION

The various embodiments herein provide an apparatus and methods for tracking movement of one or more points of a catheter by using accelerometers deployed at one or more points within the body of the catheter, including parts of the catheter deployed within the body as well as parts of the catheter deployed outside the body. The relative position of the imaging probe is then tracked by constantly monitoring the acceleration of these sensors as a function of time and using this data to calculate displacement from a baseline position.

In an embodiment method, a 2-D imaging transducer is guided into a baseline position via a catheter. The baseline position is determined and recorded. The 2-D imaging transducer captures images of the structure in interest. As the transducer is manipulated, moved and/or rotated, accelerometers disposed along the catheter and/or imaging transducer measure linear and/or rotational acceleration. Relative position is then calculated from the measure of acceleration. Meanwhile, as the imaging transducer is manipulated, moved and/or rotated additional images are captured and the corresponding calculated position is recorded. The series of images and recorded positions can then be “stitched” together to construct a 3-D image.

### BRIEF DESCRIPTION OF THE DRAWINGS

FIG. 1 shows an exemplary imaging system usable with various embodiments of the present invention.

FIG. 2 depicts a basic flow chart for processing 3-D images according to an embodiment of the invention.

FIG. 3 illustrates imaging planes of a phased array ultrasound imaging transducer when rotated about the long axis of a catheter.

FIGS. 4A and 4B illustrate access to the heart by cardiac catheterization via the femoral vein to the right atrium.

FIGS. 5A and 5B illustrate access to the heart by cardiac catheterization via the femoral vein to the right ventricle.

FIG. 6 illustrates positioning of the ultrasound sensor in the right ventricle with catheterization via the subclavian or jugular veins.

FIG. 7 depicts a catheter device consistent with an embodiment of the invention with positional sensors deployed thereon to determine relative position of the imaging transducer.

FIG. 8 depicts an exemplary B-mode ultrasound image of the endocardium.

FIG. 9 depicts an exemplary B-mode ultrasound image of the endocardium.

FIG. 10 depicts a flow chart for processing 3-D images according to an embodiment of the invention.

FIG. 11 depicts a catheter device consistent with an embodiment of the invention with rotational positional sensors deployed eccentrically within the catheter shaft to determine relative rotational position of the imaging transducer.

FIG. 12 illustrates a geometrical representation of the degrees of motion that can be registered by an ultrasonic transducer deployed on a catheter.

FIG. 13 depicts a human electrocardiogram (ECG).

FIG. 14 shows an isometric projection of the acquired image volume.

### DETAILED DESCRIPTION OF THE EXEMPLARY EMBODIMENTS

Reference will now be made in detail to exemplary embodiments of the present invention. Wherever possible, the same reference numbers will be used throughout the drawings to refer to the same or like parts.

As used herein, the terms “about” or “approximately” for any numerical values or ranges indicates a suitable dimensional tolerance that allows the part or collection of components to function for its intended purpose as described herein. Also, as used herein, the terms “patient”, “host” and “subject” refer to any human or animal subject and are not intended to limit the systems or methods to human use, although use of the subject invention in a human patient represents a preferred embodiment.

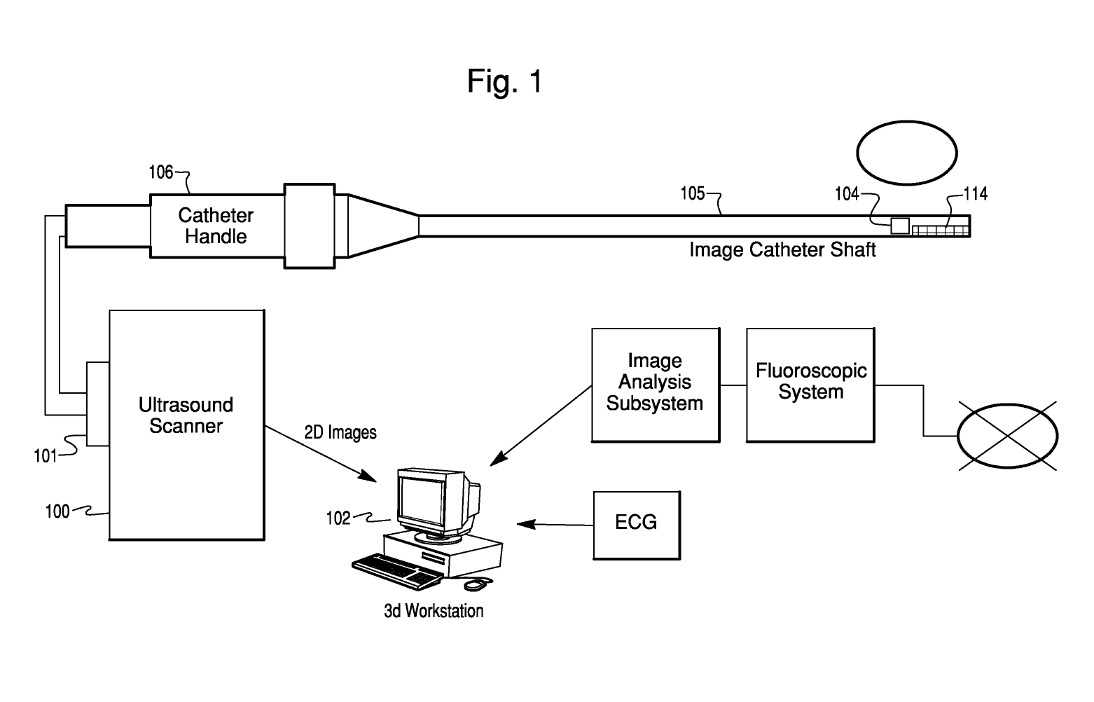

An exemplary ultrasound imaging system usable with various embodiments of the present invention is shown in the block diagram of FIG. 1. The imaging system includes an ultrasound imaging device 100, which could include within it an image processing workstation 102. The ultrasound imaging device 100 may include a display, a user interface, and an ultrasound interface all electrically coupled to a controller. The imaging system further may include a catheter handle 106 for manipulating the imaging transducer 114 disposed near the distal end of the catheter shaft 105. An accelerometer 104 can be disposed near the distal end of the catheter shaft near or in contact with the imaging transducer 114 to measure the acceleration of the imaging transducer 114. The system may further include a second accelerometer (not shown) disposed to measure the rotational acceleration of the imaging transducer 114 about the longitudinal axis of the transducer so that not only linear position, but also the rotational orientation of the imaging transducer can be calculated.

The system may further include an electrical interface 101 between the catheter handle 106 and the ultrasound scanner 100 for electrically isolating the catheter from the rest of the system to protect the patient from induced and fault currents. The electrical interface 101 can include isolation circuits on all conductors coupled to the catheter as disclosed in U.S. patent application Ser. No. 10/997,898, which published as U.S. Publication No. 2005-0124898, the entire contents of which are incorporated herein by reference. Also, the electrical interface 101 may include tissue temperature sensing and protection circuits, such as disclosed in U.S. patent Ser. No. 10/989,039, which published as U.S. Publication No. 2005-0124899, the entire contents of which are incorporated herein by reference. Additionally, the electrical interface 101 may include electrical power supply leads for each of the accelerometers positioned on the catheter, with each such power lead provided with suitable isolation circuits to protect the patient against induced and fault currents.

In certain but not necessarily all embodiments, the electrical interface 101 may include preprocessor circuits provided to perform filtering, noise reduction and/or signal integration functions. Such preprocessor circuits may be discrete circuits, such as filter circuits, or may be one or more programmable circuits, such as a microprocessor and/or digital signal processors, which are programmed and configured to perform filtering, noise reduction and/or signal integration. Such filtering, noise reduction and/or signal integration may be accomplished on signals from accelerometers positioned on the catheter, as well as on signals from the ultrasound transducers. By positioning signal preprocessor circuits within an electrical interface 101, interpreted sensor data (e.g., motion or acceleration information) can be transmitted from the interface 101 to the ultrasound scanner 100 rather than raw analog or digital signals. Transmitting interpreted sensor data may reduce the amount of data that must be transmitted to the ultrasound scanner 100, allowing use of a thinner transmission cable. Also, calibration circuitry may be included within the electrical signal interface 101 to permit calibration of accelerometers and alignment of position sensors.

The various parts that form the ultrasound system illustrated in FIG. 1 can be within the same unit, physically or functionally integrated at various levels, or one or more of these could be separately deployed as one or more separate units with various means of inter-communication of signals and control.

Various embodiments utilize a 2-D imaging transducer 114 mounted on a catheter delivery system to generate a series of 2-D images in coordination with positional information which can be used to generate 3-D image constructions. Additional information regarding rotational position or orientation of the catheter, particularly about the long axis of the catheter, can enable generation of more accurate 3-D images.

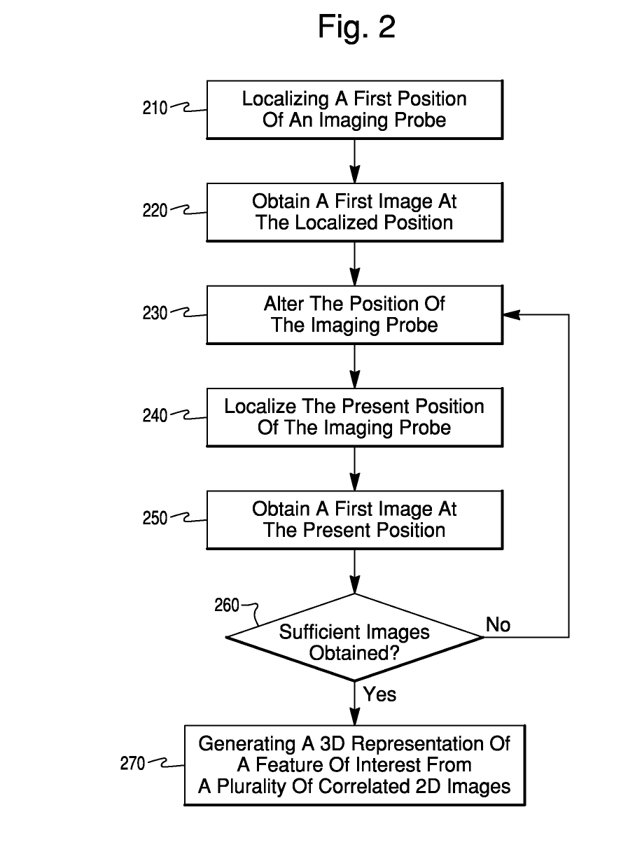

According to an embodiment as shown in FIG. 2, the system may be used to generate a still or moving 3-D representation of a feature of interest from a plurality of correlated 2-D images. Referring to FIG. 2, in step 210, sensors disposed on the imaging catheter (e.g., an intra-body ultrasound catheter probe forming part of ultrasound equipment 110) can be used to localize a position of an imaging probe (e.g., an ultrasound phased array transducer). Such a process is described in U.S. patent application Ser. No. 10/994,424 entitled “Method And Apparatus For Localizing An Ultrasound Catheter”, the entire contents of which are incorporated by reference herein.

In one exemplary embodiment for performing step 210 (and step 240), the sensors may comprise one or more accelerometers (i.e., 104), the accelerometers generate signals for locating the relative position of the imaging transducers. This embodiment positions the catheter delivery system to a baseline position near the anatomical structure of interest. This baseline position is then recorded.

Once the baseline position (or orientation) of the ultrasound imager has been localized in step 210, a first series of time-gated images is obtained in step 220 at the localized image according to various embodiments. This series of images can be stored along with the localizing information for subsequent 3-D generation to be described in detail below.

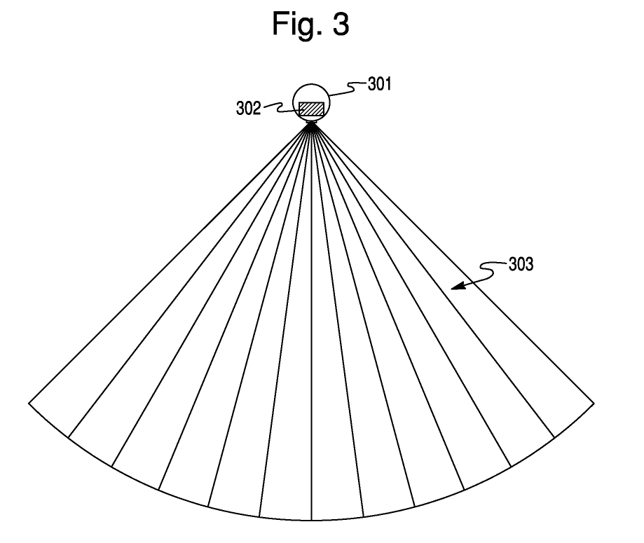

Once the series of images of the baseline position and/or rotation of the transducer array have been obtained, the position and/or rotation of the imaging catheter is altered in step 230, such as by a user rotating the intra-body catheter so that the transducer array thereon is facing a new direction. At each rotational position or step, a 2-D image can be obtained by the transducer 302 along a thin slice or plane 303, referred to herein as an “imaging plane,” as illustrated in FIG. 3. The step size or rotation angle can be selected so images cover adjoining slices or volumes (perhaps with some overlap) so that a 3-D image(s) can be reconstructed by “stitching” the 2-D slice images together, such as in the workstation 100. In another embodiment, orientation information obtained from the transducer array can be provided to the user to assist in rotating the transducer array to a proper orientation for obtaining a next imaging plane. In a further embodiment, the timing of each image slice can be controlled at least partly by use of a physiological trigger, such as a movement of a structure or an electrocardiogram (ECG) signal event. Such a physiological trigger can be matched beforehand or by user selection with a specific portion of a structure for imaging. The accelerometers 104 on the catheter can be then used to localize the present (second) position or orientation (i.e., after alteration in step 230) of the imaging transducer 106. In step 250, a subsequent image or series of images is obtained at the present location, and these images are stored along with the timing/triggering and localizing information as previously described. Steps 230, 240 and 250 can be repeated until images from a sufficient number of imaging perspectives have been obtained for 3-D rendering (see step 260). According to an embodiment, steps 230, 240 and 250 are continued even after a 3-D representation has been generated in step 270 in order to update or refresh the 3-D representation with the latest images.

A 3-D representation of a feature of interest may then be generated in step 270 by workstation 100 from the plurality of 2-D images obtained in steps 220 and 250. The ultrasonic signals produced at each position can be correlated, stitched together or otherwise matched up in accordance with known techniques.

Thus, a first step involves localizing the imaging transducer at a baseline position near the structure of interest. Although the method and apparatus of the invention can be used to generate 2-D and 3-D images of virtually any anatomical structure, the invention is described herein with reference to embodiments used to generate 2-D and 3-D images of the endocardium and its sub-structures. Accordingly, exemplary reference will be made to the endocardium and its sub-structures. To acquire images of the endocardial surface of the right and left ventricle, a phased array ultrasound imaging catheter is positioned within the heart via percutaneous cannulation using standard cardiac catheterization techniques of the femoral vein or the subclavian or jugular veins.

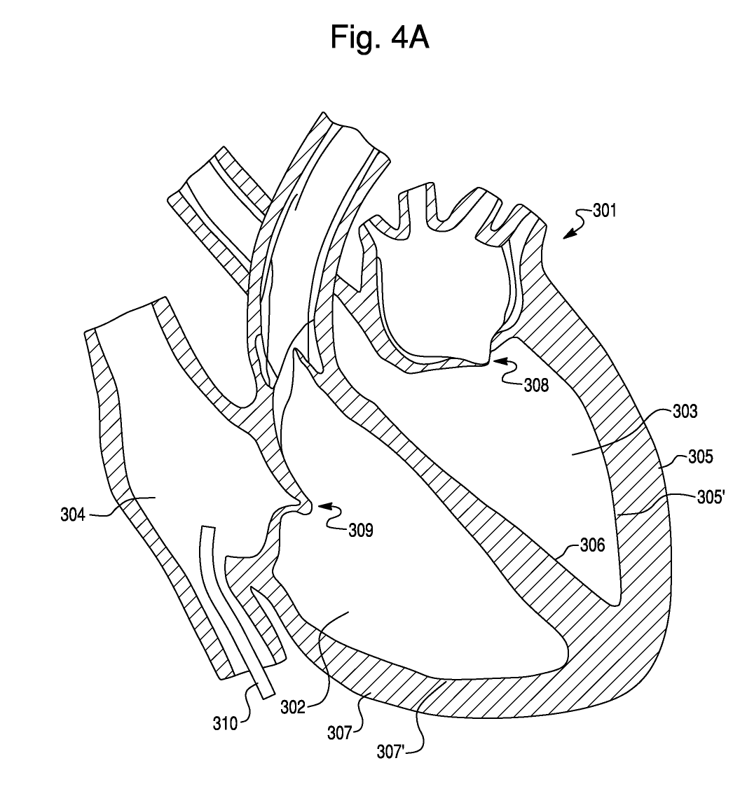

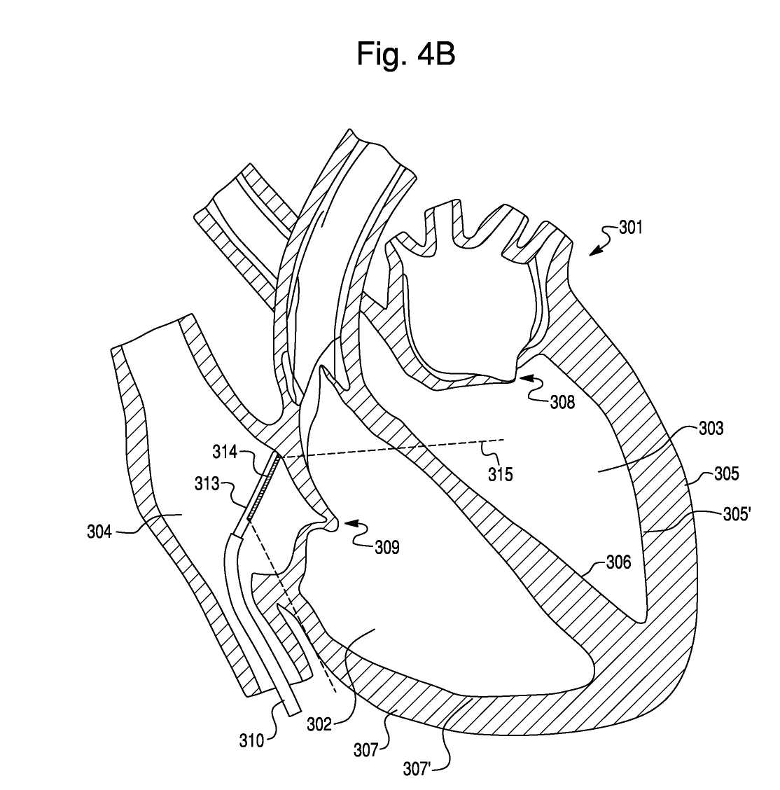

In order to properly position the phased array ultrasound imaging catheter, a long preformed intravascular sheath 310 can be advanced under fluoroscopic control into the various chambers of the heart 301, as shown in FIG. 4A. The sheath may be sufficiently transparent to ultrasound or has an ultrasonic deployable window. FIG. 4A illustrates access to the heart by cardiac catheterization via the femoral vein to the right atrium 304. A guide wire 311 (shown in FIG. 5A) may be used to properly position the sheath in or near the orifice of the tricuspid valve. Once the sheath is positioned with its distal end in the right atrium 304, the phased array ultrasound imaging catheter 313 can be advanced through the sheath until the ultrasound transducer 314 is properly positioned outside the tricuspid valve 309 for imaging the right ventricle 302, as shown in FIG. 4B. In this position, the field of view 315 (indicated by dotted lines) of the ultrasound transducer 314 can address most, if not all of the right ventricle 302, right ventricle wall 307 and much of the septum 306.

For imaging the left ventricle 303, the ultrasound transducer 314 needs to be positioned within the right ventricle. This can be accomplished by passing a guide wire 311 through the sheath 310, and under fluoroscopy control, passing the guide wire 311 through the tricuspid valve 309. The sheath 310 is then directed over the guide wire 311 into the orifice of the tricuspid valve 309 and advanced into the right ventricular cavity 302, as illustrated in FIG. 5A. Once the sheath 310 is properly positioned, the guide wire 311 is withdrawn and the phased array ultrasound imaging catheter 313 is advanced under fluoroscopic control through the sheath 310 into a position inside the right ventricular in the mid cavity region, as illustrated in FIG. 5B. In this position, the field of view 315 (indicated by dotted lines) of the ultrasound transducer 314 can address most, if not all of the left ventricle wall 305 and much of the septum 306.

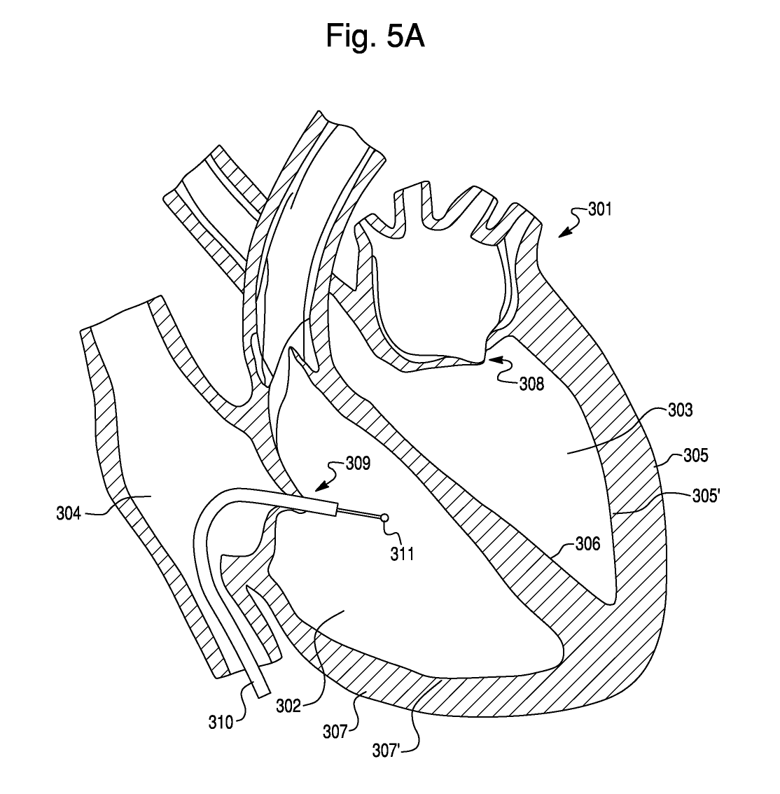

Other methods other than the use of a sheath 310 to position the ultrasound phased array catheter 313 in the heart 1 can be employed. For example, a steerable ultrasound catheter, such as disclosed in U.S. Patent Publication 2005/0228290, can be used and guided directly under fluoroscopy control into position within orifice of the tricuspid valve or within the right atrium, as illustrated in FIG. 6. FIG. 6 illustrates positioning of the ultrasound sensor 314 in the right ventricle 302 with catheterization via the subclavian or jugular veins.

In order to be able to correlate and stitch together multiple 2-D images to render a 3-D reconstruction, the position of the imaging probe, such as an ultrasound phased array, at the instant a 2-D image is obtained needs to be accurately. Various methods for localizing a catheter within the body are disclosed in U.S. application Ser. No. 10/994,424 incorporated by reference above. However, in a moving organ such as the heart, forces from flexing muscles and flowing blood can cause the catheter to move about rapidly. Such movements may have a shorter interval than the position-resolving time of known positioning methods. For this reason, additional positional information may need to be used to narrow the position error on 2-D images sufficient to render clear 3-D images. Thus, fast responding position or movement sensors may be added to the catheter to supplement or update imaging probe position information.

In various embodiments of the present invention, accelerometers are added to the catheter in the vicinity of the imaging probe to obtain supplemental position information. Accelerometers have a short time constant (i.e., acceleration is sensed very quickly) and displacement is calculated by double integrating the sensed acceleration over time, and thereby provide near instantaneous positional information. Since errors will build up in the calculation of position based upon measured accelerations, such position information is best used in combination with (such as supplementation of) periodic baseline positions obtained by other methods.

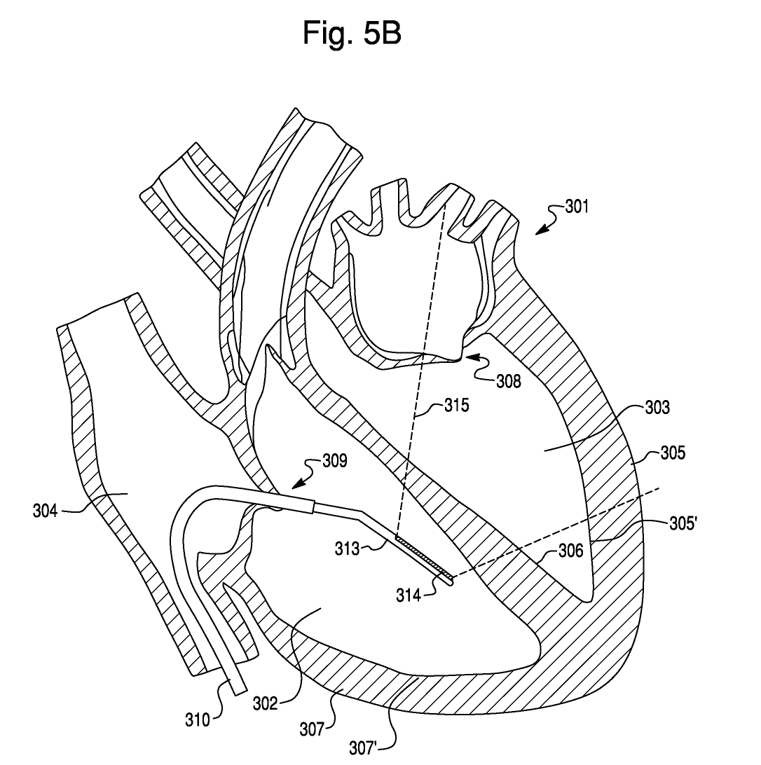

Various embodiments include linear and rotational position sensors which may comprise one or more accelerometers deployed near or in contact with the imaging tip. In the embodiment shown in FIG. 7, the catheter 613 has an imagining transducer 614 and is guided into place by sheath 610. Deployed near the imaging tip is an accelerometer 604, which is capable of detecting the acceleration of the imaging tip. The accelerometer 604 can be configured to be in rigid contact with the imaging tip such that any displacement of the tip will lead to the displacement of the transducer. Another accelerometer 603 can also be employed within the body of the catheter configured to sense rotational displacement of the imaging tip around the long axis of the catheter tip.

Once a known baseline position is established, changes in the position of the catheter can be detected by the accelerometers sensing acceleration of the catheter. According to Newtonian physics, acceleration is the derivative (with respect to time) of an object's velocity and the second derivative (with respect to time) of the object's position. Accordingly, by calculating the second integral of the sensed acceleration over a time interval will yield an estimate of the relative position of the accelerometer (i.e., relative to the position before the time interval during which the acceleration was applied). In this manner, accelerometer data can be used to locate the position of the catheter tip, and thus the phased array, in combination with other sensors of position. Generally, accelerometers are useful for detecting the movement of a sensor, since they detect only the acceleration at the point of the sensor. While accelerometer data can be used to determine velocity, and change in position over time, that information needs to be combined with some reference or relative measurement of position at some starting point of time. Then the instantaneous position of the accelerometer (and by association the sensor) can be confirmed by performing the integral function of acceleration and adding the result to the reference position using vector addition.

The calculation of the double integral of sensed acceleration to yield displacement can also be done by successive additions over short time intervals (e.g., one or a few milliseconds). Such calculation methods may have advantage since the accelerometer output can be digitized. Also, in a dynamic environment like the heart, acceleration will vary constantly.

By including three or more accelerometers positioned about the catheter so they sense accelerations along three different axes, accelerations in all three dimensions can be measured. The accelerometers may be oriented on the catheter and configured to sense accelerations along three orthogonal axes, or along three different but not necessarily orthogonal axes. Alternatively, a single accelerometer unit may include acceleration sensors oriented along different axes and configured to output three-dimensional acceleration data. By performing a double integral on the measured accelerations in each dimension over a time interval, a three dimensional displacement vector can be obtained for the interval. This displacement vector may be saved in memory or added to the baseline three dimensional positional coordinates and stored in memory in real time as an instantaneous position vector. Alternatively, the acceleration data may be stored in memory for later use by the processor in calculating instantaneous sensor positions associated with each 2-D image during post processing.

A number of techniques exist for detecting the baseline position of the catheter within the heart at a particular instance. These include fluoroscopy in which the catheter is directly imaged by X-rays taken in one, two or more planes. Disclosure of methods of locating an ultrasound imaging catheter using fluoroscopy is provided in U.S. patent application TBD, entitled “Catheter Position Tracking using Fluoroscopy and Rotation Sensors”, filed concurrently with the present application and incorporated herein by reference in its entirety. Other methods of locating the catheter employ the use of sound (i.e., echo location) or magnetic fields to measure the position using triangulation methods. Examples of such position sensors and methods are disclosed in U.S. application Ser. No. 10/994,424, entitled Method And Apparatus For Localizing An Ultrasound Catheter, filed Nov. 23, 2004, incorporated by reference above. Since the accuracy of position estimates based on accelerometer data degrades with time due to the accumulation of errors, frequent measurements of catheter position using absolute or relative position measuring methods may be desirable. In such embodiments, the relatively slow process of determining catheter position using absolute position sensors, such as fluoroscopy or echo-location, is supplemented by rapid position change measurements using accelerometer data.

Existing techniques for measuring a baseline position can be used in combination with position information obtained from the accelerometers to more accurately locate the instantaneous position of the accelerometer, and thus the nearby sensor. Specifically, the instantaneous position can be estimated as the vector sum of the latest baseline position and the displacement from that position calculated as the double integral of the acceleration measured since the time when the baseline position was measure.

Suitable accelerometers are capable of sensing accelerations on the order of 35 degrees per second² angular acceleration and about 10 cm per second² linear acceleration. Examples of suitable accelerometer for use in one or more of the positions on the catheter include micro-electromechanical sensor (MEMS) accelerometers, such as disclosed in U.S. Pat. No. 7,104,130 and carbon nanotube accelerometers such as disclosed in U.S. Pat. Nos. 6,445,006 and 6,946,851, and U.S. Patent Publication No. 20050179339. All of the foregoing example references are incorporated herein by reference in their entirety.

Signal data from the accelerometer can be communicated down the catheter using a shielded cable to limit cross-talk interference from the ultrasound transducer signals. A suitable cable for this purpose is the miniature coaxial cable used for connecting each of the ultrasound phased array elements to the ultrasound system.

Acceleration measurements by the accelerometers can be communicated to the system computer, such as through a serial port or a USB port from the accelerometer interface circuitry. The system computer can then use the acceleration measurements to determine position data according to various methods disclosed herein.

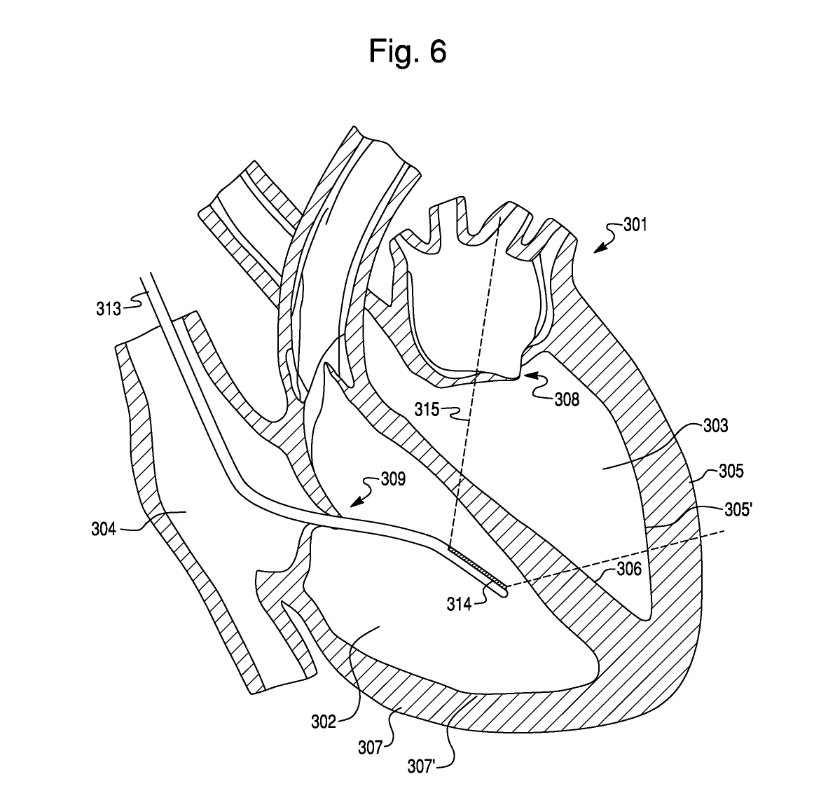

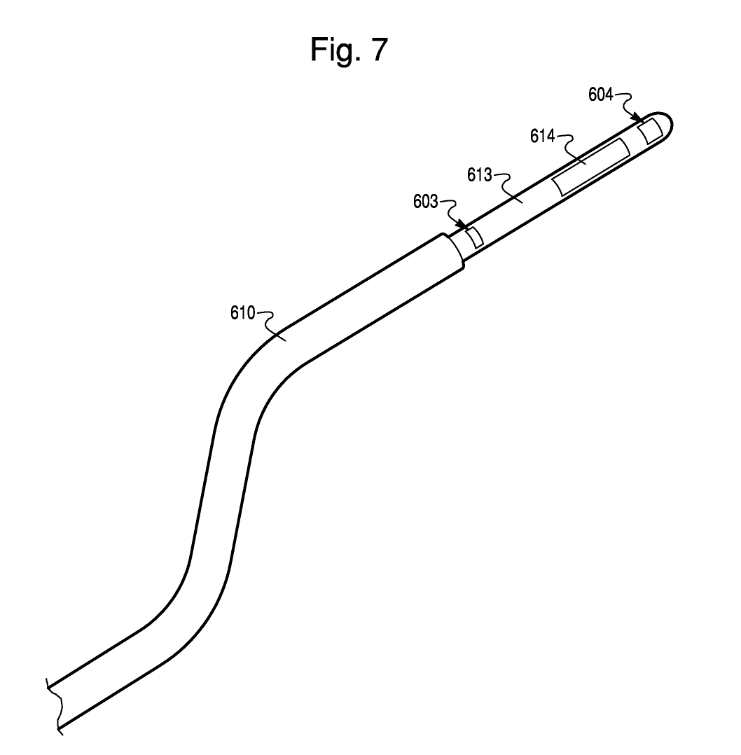

An ultrasound phased array transducer operated using B-mode ultrasound imaging technique renders 2-D images, such as the images of the left ventricle of the heart illustrated in FIGS. 8 and 9. B-Mode ultrasound imaging displays an image representative of the relative echo strength received at the transducer. A 2-D image can be formed by processing and displaying the pulse-echo data acquired for each individual scan line across the angle of regard 15 of the phased array transducer. This process yields an approximately two-dimensional B-mode image of the endocardial surface of the ventricle, examples of which are illustrated in FIGS. 8 and 9. The cardiologist may define the edge of the endocardial surface 5′, 7′ in the image by manually tracing the edge using an interactive cursor (such as by using a trackball, light pen, mouse, or user-interface device) as may be provided with the ultrasound imaging system. By identifying the edges of structures within an ultrasound image, an accurate outline of ventricle walls can be obtained and other image data ignored. The result of this analysis may be a set of images and dimensional measurements defining the position of the ventricle walls at the particular instants when the ultrasound images are obtained within the cardiac cycle. The dimensional measurements defining the interior surface 5′ or 7′ of the endocardium can be stored in memory of the ultrasound system and analyzed using geometric algorithms to determine the volume of the ventricle.

The system computer can combine the position information from the 2-D ultrasound images acquired from the ultrasound scanner (100) with the ECG data obtained through any of the means currently known in the art. The ECG signals, which generally correlate to the phase of the heart beat within the cardiac cycle, can be used to judge whether the image frames acquired over a number of cardiac cycles correspond to relatively similar mechanical states of contraction or relaxation from one acquired frame to the next acquired frame. Methods for using ECG signals to combine images and average multiple images at a particular phase or relative time within the cardiac cycle (time gating) are described in U.S. application Ser. No. 11/002,661 published as U.S. Patent Publication No. 2005/0080336, the entire contents of which are incorporated herein by reference in their entirety.

Correlating ultrasound images to phases in the cardiac cycle can be important since otherwise, different parts of the reconstructed 3-D image might be obtained from different parts of the cardiac cycle and provide an inaccurate representation of the cardiac structure being imaged. However, care has to be taken to ensure that there exists no appreciable time-difference between the ECG signals and the ultrasonic images, since such delays could be entrained when dealing with large amounts of image data as compared to the relatively low density of data from a few ECG channels.

Additional system components may be provided as would be readily apparent to one of ordinary skill in the art after reading this disclosure.

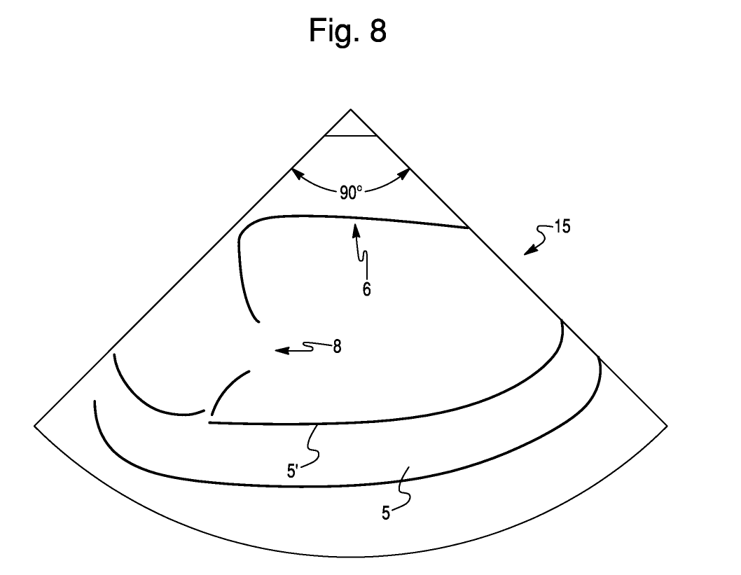

An embodiment of a method of processing 2-D ultrasound images according to an embodiment of the present invention is illustrated in the flowchart of FIG. 10. It should be appreciated that, as with other methods described herein, the method shown in FIG. 10 may be implemented using the exemplary imaging system of FIG. 1, or using another suitable imaging system.

Referring to FIG. 10, in step 900 the imaging system obtains the ECG of the subject under investigation. The relative phase of the cardiac cycle is judged from the ECG and, if appropriate (905), as described in this disclosure, the corresponding image along with the position data of the ultrasound transducer is obtained (925, 910, 915, and 920). The ultrasound image referred to herein can include image data in various formats. The data can also originate from one or more parts of the ultrasound image processing chain, including but not limited to RF data, pre scan conversion data, or scan-converted digital data, etc., as would be apparent to one of ordinary skill in the art from reading this disclosure. The above technique can also include continuous collection of ECG, image, and position data followed by later selection of useable data, based on the cardiac cycle, from such a collection during post processing.

The acquired data can then be arranged dynamically in a 3-D data matrix with appropriate recalculation based on positional information (930). Any number of algorithms may be used for accomplishing this. Generally, the image data will be stored in memory. Time and ECG phase information can be stored with the images or correlated to the images so that each image can be matched to a particular time in the cardiac cycle. Additionally, the sensor position information associated with each image can be stored in memory, either with the image or so that it can be matched to the images. As mentioned above, the sensor position information may be stored as a series of three dimensional vectors (i.e., the instantaneous position information), as a series of baseline positions and a series of displacement vectors, or as a set of baseline measurements and accelerometer measurements which can later be processed to derive the instantaneous position of the sensor. The resulting data set may be a data table of images, ECG base information, and corresponding sensor positional information (i.e., the position of the sensor at the time each image is obtained). Alternatively, the images, ECG. and positional information may be stored as three separate data tables that are linked by means of an index or a pointer array.

As an alternative to linking images to instances within the cardiac cycle, ultrasound images may be obtained at particular periods during the cardiac cycle. For example, when the heart is briefly at rest between beats, a number of ultrasound images may be obtained and combined or averaged. In such a method, ECG signals may be monitored in order to schedule or trigger when the ultrasound images are obtain. Such ECG signals may include, for example, the R wave and the ST wave. In an example embodiment, when the R wave is detecting in the ECG signals, a series of ultrasound images may be initiated, with the rotational orientation of the transducer array changed (e.g., by a miniature stepping motor) between each image. With each rotation, the new orientation of the transducer array can be determined using the methods and structures of the various embodiments. By rapidly repeating the steps of imaging and rotating the transducer array until the ST wave is detected, a scan of 2-D ultrasound images can be obtained through the angle of rotation of the imaged portion of the heart while it is at rest. This sequence of 2-D images may then be combined by the system processor using the determined transducer orientation information as a reference point.

In the various embodiments in which the transducer array is rotated between ultrasound images in order to obtain a set of images for generating 3-D images, the imaging can be conducted as a series of position measurements and image recordings. For example, at the start of a phased scan, a baseline precise position and orientation of the transducer array may be obtained according to the various embodiments. An ultrasound image may then be obtained, with the image data recorded along with the precise position and orientation information. The transducer array may then be moved, such as rotated through a small angle before taking the next ultrasound image. The position and orientation of the transducer array may be determined using accelerometer measurements according to various embodiments to enable the system to measure or determine the position/orientation with respect to the baseline position. With each ultrasound image, the image data and precise position and orientation data may be stored together so that the system can then combine the images into a 3-D or 4-D image. This process of rotating, determining precise position and orientation, imaging and storing the image and position/orientation data may be repeated through a desired angle of rotation (e.g., to view a desired region of the heart), for a set amount of time, or between various points in the cardiac cycle, such as may be determined by detecting different waves within ECG signals.

With the image, ECG and position information stored in memory, the processor can correlate, combine or average images of a particular point in the cardiac cycle. This may be accomplished by reorganizing the data table, or, more likely, including metadata which allows the processor to quickly identify images at common points in the cardiac cycle. Similarly, the data may be processed to correlate or align images according to their positional information.

In another embodiment, edge-detecting image processing algorithms can be employed to recognize the surfaces of structures within the heart and store positional information for the recognized surfaces. Such techniques determine the edge of a structure by noting a sudden rise in brightness across a short distance. When the processor recognizes that a surface exists at a particular location, the image data can be stored as the positional (X, Y, Z) measurements of the location (e.g., as a 3-D vector), rather than as image pixel data. This process can thus generate a 3-D surface dataset. With recognized surface datasets from each image stored in memory, the processor then can stitch together the surface datasets using ECG and sensor positional information in order to render a complete 3-D image data set over the cardiac cycle.

When all required frames are collected (steps 940, 935; with the former being defined directly or indirectly by the user), a 3-D representation of the volume being imaged can be generated in step 950. A number of different algorithms may be used to generate a 3-D representation of the heart based on the image data. As an example, the transducer positional data and the distance and angle measurements within the images can be used to build up a 3-D positional dataset representing the detected structures of the heart. Since the heart is continually in motion during the cardiac cycle, this process of building up a 3-D positional dataset should be only performed at particular points within the cardiac cycle, as indicated by the correlated ECG data. The result can be a series 3-D positional data sets (vectors) defining the imaged heart structures at particular points within the cardiac cycle. By repeating this process for all times within the cardiac cycle, the combined database can provide a 4-Dimensional (4-D) image of the heart over the entire cardiac cycle. The 4-D dataset may be relative to a particular point on the heart or transformed into another frame of reference, such as with respect to the body of the patient.

Once a 3-D or 4-D dataset has been generated by the processor, the information can be represented on any appropriate user interface in step 955. This may be accomplished by any number of display algorithms. For example, the processor can map the 3-D information into 2-D display by means of raster graphics techniques.

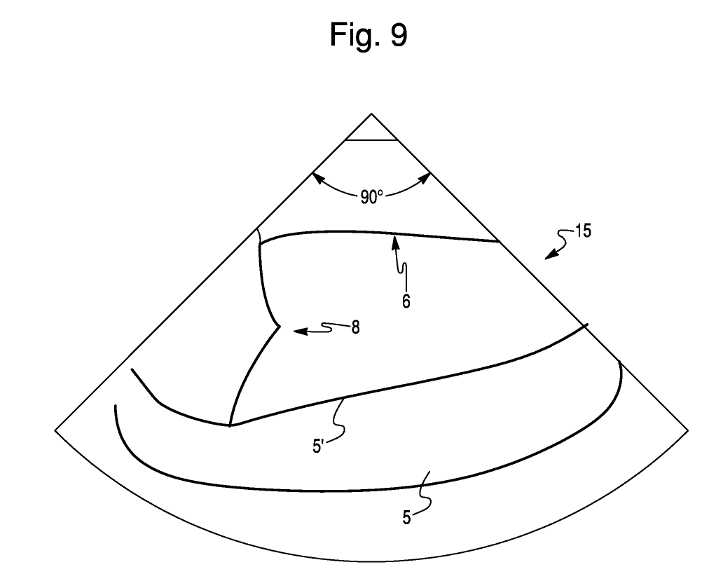

As mentioned previously, one of the challenges facing such reconstructions is the determination of the rotational orientation of the imaging catheter about its longitudinal axis. As shown in FIG. 11 (105), the rotational orientation and linear position of the shaft of the catheter dictates the rotational orientation and linear position of the imaging array in the case of catheters with sufficient torsional stiffness to allow a 1:1 rotation between the tip and the base of the insertable part. FIG. 11 also discloses one such position sensing technique, wherein a linear accelerometer is deployed eccentrically within the catheter handle. This is illustrated in FIG. 11 where accelerometer 1004 is positioned close to a surface of the catheter and thus away from the catheter centerline which defines a longitudinal axis of rotation. When the catheter 1005 is rotated about its longitudinal axis, the accelerometer 1004 will sense the rotation as accelerations both parallel and perpendicular to the catheter surface.

FIG. 11 depicts a cross sectional view of a catheter shaft 1005. An accelerometer 1004 is eccentrically deployed within the catheter shaft 1005. In this manner, the accelerometer 1004 is capable of sensing the rotational acceleration of the transducer array about its centerline/longitudinal axis of rotation, enabling a processor to derive the rotational position from a linear accelerometer deployed in this fashion. By initially aligning the rotational accelerometer 1004 with the imaging transducer 1014, as the rotational orientation of the imaging transducer changes, so does the rotational accelerometer 1004. Relative rotational position or orientation can again be determined by taking the second integral of the resulting rotational acceleration function over a particular brief interval of time.

A rotational velocity or acceleration sensor can also be deployed concentric to the shaft of the catheter to obtain the rotational motion information. Optical or magnetic position tracking sensors can be used to serve this purpose. An optical sensor may include a photo sensitive diode that can translate sensed changes in brightness into electrical signals. Markings, such as light or dark banding spaced at regular intervals, can be provided on the catheter or the sheath which can be sensed by the photo sensitive diode to provide information regarding the rotation position of the catheter with respect to the sheath. The optical sensor can also include a light source, such as a light emitting diode, so that the sensor can be self contained. An optical sensor can determine the speed and extent of rotational motion by counting the number of light or dark bands that pass over the photos sensor. One or more of the bands may have a particular characteristic (such as greater or less width) to permit the sensor to sense a particular orientation. Then rotational position can be determined by counting the number of bands that have passed over the photo sensor since a particular orientation band was sensed.

A magnetic position tracking sensor can include a magnetic field sensor, such as a sensor used in a disk drive sensor head, and regularly spaced ferromagnetic material markings. For instance, the catheter may include a magnetic field sensor and the sheath may have bands of ferromagnetic material positioned at regular intervals about the circumference. A magnetic position sensor can detect the rotation of the catheter within the sheath by counting or otherwise sensing passages of the ferromagnetic bands over the sensor. As with accelerometers, signals from the optical or magnetic rotational position sensors can be provided to the system computer by means of cabling and standard connectors.

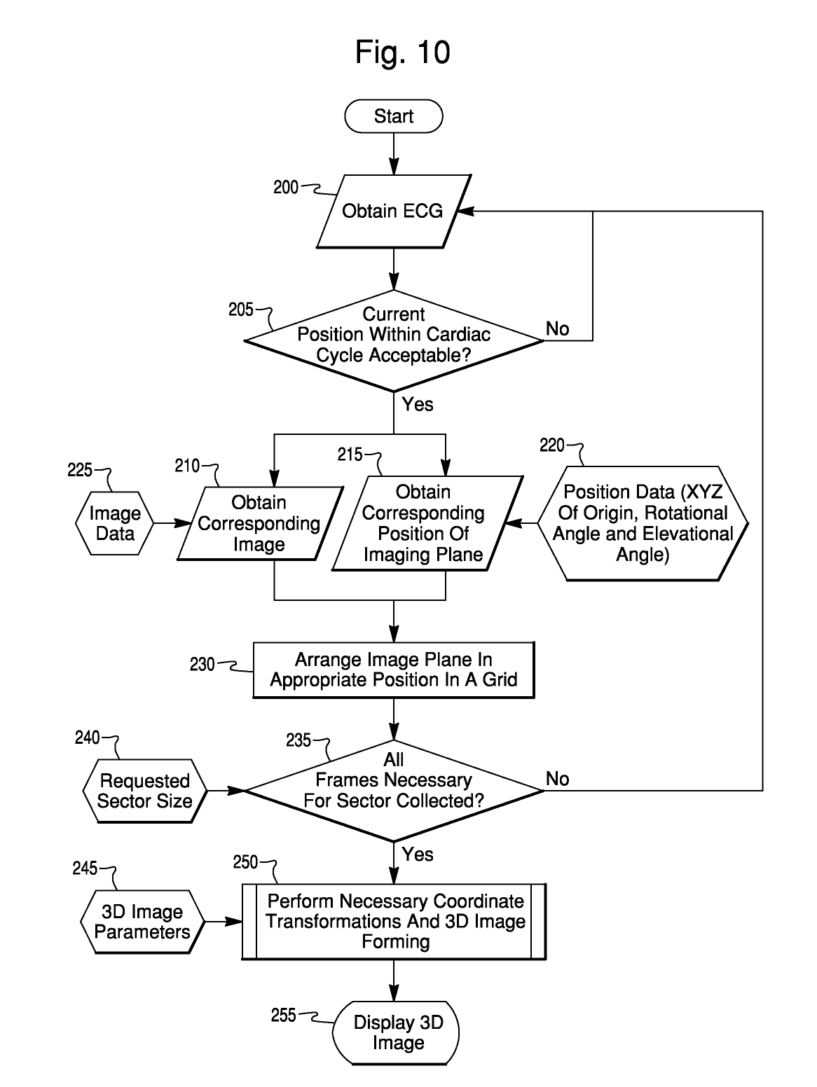

FIG. 12 shows a geometrical representation of the degrees of freedom (position and orientation) that can be registered by an ultrasonic transducer 1103 deployed on a catheter 1101. A side-firing phased array 1103 is shown in FIG. 12 with the catheter deployed at the tip of the imaging catheter. Although a side-firing phased array transducer is shown in FIG. 12 with the catheter deployed at the tip of the catheter, other imaging formats and transducers, including circular arrays, mechanically scanned transducers and other transducers can benefit from this invention. Further, transducers can also be deployed in the body of the catheter and not necessarily at the tip.

In FIG. 12, the direction D represents the mid-line of the 2-D imaging plane (shown in the plane of the image) which extends along a plane parallel to the length of the array and perpendicular to the face of the sound emitting faces of the array elements. As shown, the transducer tip is capable of being located in 3 dimensional space (represented by x′, y′, z′). The base of the transducer can also be located in space through x, y, z dimensions. Further, the transducer is capable of being rotated through an angle of θ, around its longitudinal axis. More broadly, the transducer array may move through six degrees of freedom that may need to be accounted for in constructing and combining images. Specifically, the array may be positioned in 3-dimensional space of left/right, up/down and in/out (with respect to the long axis of the catheter), rotated through angle θ (roll angle) and oriented so the linear array is tilted up/down (inclination or pitch angle) and angled left/right (yaw angle). In order to measure motions along each of these degrees of freedom, a number of accelerometers may need to be included and data from each integrated with others to accurately track the position and orientation of the array. For example, X and Y axis accelerometers on each end of the array may be used to sense both position (i.e., left/right, up/down) and orientation (i.e., inclination or pitch and yaw) of the array while a single axial accelerometer provides in/out position data and a rotational accelerometer provides roll angle data.

In another representation, the origin of the imaging midline (D), can be located in space (e.g., x,y,z). The inclination, yaw and rotation of the imaging plane can then be measured against any arbitrarily assigned axis as rotation θ, and inclination Φ, as well as yaw angle (not shown in FIG. 12 as this motion is with respect to the plane of the image). From either of the above three coordinate definitions of position, the relative positions of the imaging planes, can be easily identified as the transducer is rotated and moved through space. This positional information then can be utilized in image processing, such as shown in the embodiment illustrated in the flow chart in FIGS. 2 and 10.

The accelerometers and position sensors described above provide information necessary for the processor to solve the geometric relationships in order to locate the transducer in 3-D space. Linear motion sensors on the catheter which sense the axial deployment of the catheter within the sheath can provide Z axis position information with respect to the sheath. Rotational sensors can provide the rotation angle θ data with respect to the catheter. Accelerometers near the tip (i.e., the distal end) of the transducer array can provide X, Y and Z and/or Φ acceleration data that can be used to calculate displacements along these dimensions. Optionally, two or more accelerometers positioned near the proximal end of the transducer array can provide X and Y information which combined with positional data from accelerometers near the distal end can be used to calculate inclination and yaw orientation and displacement of the linear array with respect to arbitrary reference axes.

The position sensors continuously monitor either position or a change in position in rotation as well as translation of the catheter shaft and provide this information to the processor. Whenever a change in position or acceleration is detected by the sensors, the processor updates the relative position of the catheter shaft either as a rotation, as a translation or as a combination of the two.

Just as the process of reconstructing 3-D images from a series of 2-D images requires knowing the transducer array position and orientation information for the six degrees of freedom, position errors must be accounted for in each of the six dimensions. Errors in each dimension of position or orientation information combine to yield the total positional error of each ultrasound image. Further, a combined image will have positional errors of features that are combinations of those of the component images. Such errors result from accelerometer sensitivity and cumulative integration errors.

Alternatively, the calculation of position can be simplified by using a catheter that can be held in predictable positions. Provided by either designing a catheter that has a high flexural stiffness through rotation spanning at least the volume of interest, or by limiting the flexural movement of the imaging array by encapsulating it within a mechanically limiting outer sheath with an acoustical window through which a clinically acceptable image can be obtained. In this manner, the processor need not account for the flexure of the catheter. Thus, a measure of rotation obtained at the base of the catheter can be extrapolated to determine the positional orientation of the tip of the catheter several inches away. According to one embodiment, the imaging catheter is introduced through a long sheath with favorable acoustic properties. The ultrasonic image formed through this sheath enables a clinically acceptable ultrasound image throughout the volume of interest.

In another embodiment, position sensors are deployed in or near the transducer located on the catheter which allow, either directly or indirectly, tracking of the spatial position of the acoustic array. Examples of suitable echo location position sensors are disclosed in U.S. application Ser. No. 10/994,424, previously incorporated by reference. As described earlier, such tracking need not necessarily include rotational position. In such instances, a combination of the rotational position tracking disclosed herein (e.g., utilizing accelerometers deployed either eccentric to the axis of the catheter or rotational accelerometers deployed concentrically to the axis of the catheter, or optical or magnetic rotational sensors) along with the position tracking of the acoustic array, by magnetic, acoustic, or electrical means, can be combined to obtain the necessary data to enable the processor to accomplish 3-D imaging according to the various embodiments.

The frequency of baseline position measurements, accelerometer provided displacement measurements, rotational position estimations using any of the techniques previously described, and the imaging frame rate may need to be sufficiently high to provide the degree of resolution required by the particular diagnostic objective for the examination. Further, the positional (baseline measurements plus instantaneous displacement estimates) and rotational measurements and imaging may need to be timed such that all three of these measurements/estimations are within an acceptable time-span or time-correlation error band to permit clinically acceptable 3-D representation. This latter concern may arise because the duration required to record each measurement or image may be different and there will be an error (i.e., degree of uncertainty) associated with the time at which each measurement is obtained. If such errors are not properly managed or otherwise taken into account, the result may be a blurring the generated 3-D images. In this regard, the short measurement time of accelerometers may be used to provide an estimate of instantaneous position that closely matches the time of each 2-D image.

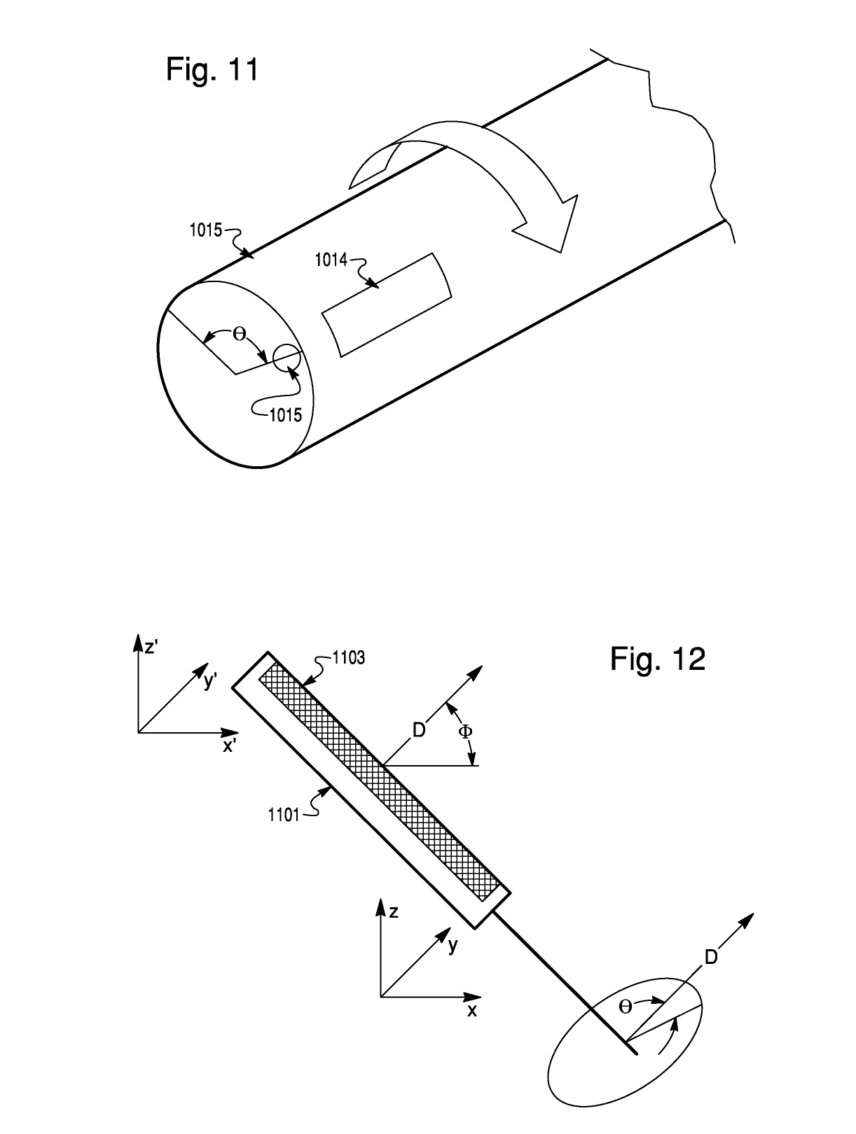

FIG. 13 shows a representation of the human electrocardiogram (ECG). Highlighted sections show areas of ventricle diastole, where the cardiac muscle activity is minimal. It is at the end of this phase of the cardiac cycle that the ventricles of the heart are at their maximum volume. More precisely, during Diastole the ventricles relax as the ventricle muscles repolarize (evidenced by the T wave) and enlarge as the atria are emptying into the ventricles. The P wave corresponds to the depolarization of the atria by the Sinoatrial (SA) node, which causes a last squeeze in the atria to push the remaining blood into the ventricle. This is called atrial systole. Thus, the ECG signal provides important timing information related to the shape and motion of the heart chambers. Images acquired during the ventricle diastole phase of the cardiac cycle are useful for reconstructing 3-D images of the heart, since a maximum number of ultrasound images can be acquired of the ventricle in the relatively longer time-spans during which the heart assumes a particular shape. In cases where abnormal conditions exist, such as rhythm abnormalities, and where such abnormality is atrial fibrillation in particular, such periods of mechanical inactivity might be too short or even absent. In such situations, multiple frames may need to be acquired and averaged to reduce the overall spatial error in the estimation of the 3-D image.

By limiting ultrasound imaging to the time between heart beats, from the end of the repolarization of the previous beat to the start of the next depolarization, as illustrated in FIG. 13, distortions and artifacts in the catheter location estimates caused by muscle movement may be avoided. By reducing distortions and artifacts in catheter position caused by muscle movement, sequences of ultrasound images can be more accurately combined to generate 3-D images of the heart. If the heart is assumed to be relatively still (i.e., static), then overlapping images can be easily stitched together to generate a 3-D image.

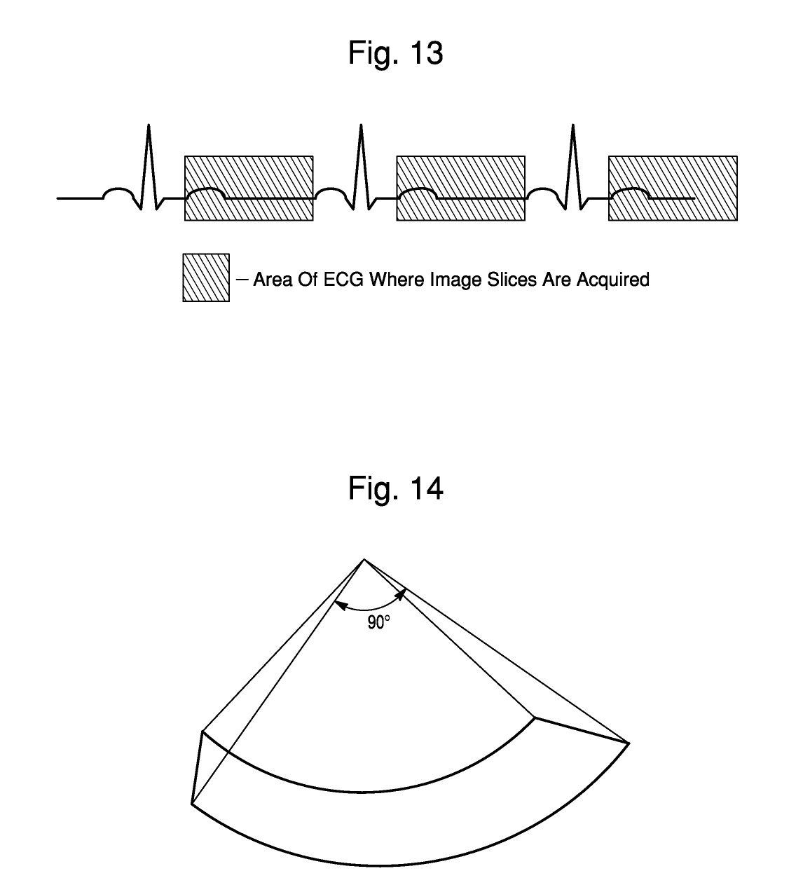

FIG. 14 shows an isometric projection of the acquired image volume, assuming that the underlying imaging technique is a phased scan. However, other scans such as linear scans and circular scan profiles can also be employed in alternative embodiments.

The aforementioned embodiments assume that the underlying imaging is carried out through what is generally known in the industry as B-mode imaging. Additional embodiments include 3-D reconstruction of Color Doppler data, with data separation between underlying tissue and Color Flow information and with the possibility of data separation between different flow directions. Such embodiments use the same methods as the embodiments described above for locating the instantaneous position of the imaging sensor except that the image data is obtain in M-Mode or Color Doppler mode.

Still further embodiments limit the catheter to translational and rotational movements only, such as by deploying it within a long sheath. In such embodiments, only 2 degrees of motion (i.e., rotation and translation) need be measured so the system need only include 2 accelerometers in the body of the catheter to enable accurate instantaneous position determination. Any translational movement in such a mechanically limited embodiment would still yield imaging planes similarly disposed, however, with vertical offsets (along the length of the catheter) from one to another, depending upon the relative translational positions of the catheter as each of the image frames is captured.

A further embodiment is suitable for instances where the body of the patient, and hence the tip of the catheter, is expected to move in a rhythmic fashion, such as breathing. In this embodiment, filtering techniques common in the ultrasound imaging art can be applied to the position estimation to compensate for such rhythmic and predictable motion. In a further embodiment suitable where the body of the patient, and hence the tip of the catheter, is expected to undergo random motion, one or more reference accelerometers or position sensors can be attached to the patients body. Acceleration or displacement measurements from this body sensor can then be subtracted (or otherwise removed) from the catheter movement measurements, thus yielding actual catheter movement with respect to the patient's anatomy. Such embodiments can be used to generate 3-D and 4-D image datasets that are correlated to a body-centric frame of reference. Additionally, the use of accelerometers or position sensors positioned on the body of the imaging subject to gather movement measurements that can be used to account for breathing and other subject movement artifacts. Additional sensors may be useful for calibrating and calculating the acceleration, speed, displacement and position information from catheter-based accelerometers. Further, information from catheter-based accelerometers, both subcutaneous and external (e.g., on or near the catheter control handle) may be combined with information from the body accelerometers or position sensors in order to cross-correlate and cross-calibrate the sensors for measuring or calculating acceleration, speed, displacement and position information. Thus, breathing and other movement induced accelerations can be effectively subtracted from the measured catheter accelerations in order to enhance the accuracy of the determined position of the catheter position with respect to the subject.

Many of the foregoing embodiments that enable a 3-D reconstruction of ultrasound images of the heart are best suited to a heart with a rhythmic cardiac cycle. However, generating a 3-D reconstruction of the heart may be particularly useful when the patient is in Atrial Fibrillation or flutter since some causes of such conditions may be deduced from ultrasound images. In such situations, the heart is flexing in irregular and unpredictable patterns that may be disjoint from the ECG patterns. In such conditions, methods that use ECG signals to assist in forming a 3-D image may be infeasible since the position of the heart walls may be unpredictable or the ECG pattern is erratic. This may be true especially in conditions of Atrial Fibrillation when the motions of the atrial walls are random, although small. Under such conditions, 3-D reconstruction of ultrasound images may be accomplished over small regions by quickly imaging, rotating the transducer array and imaging again. Images taken closely together in time may then be combined to render a 3-D image through a narrow angle of rotation.

In case of a magnetic substance being used as an accelerometer element, including electrically excited magnets (e.g., electromagnets), deployment of permanent or electromagnets in the vicinity of the patient may be used to enhance the sensitivity of the accelerometers. Such magnets may be positioned external to and in the vicinity of the patient or within or on the catheter body itself

In yet another embodiment, the precise position and orientation information obtained by the various embodiments may be combined with information obtained from other sensors or patient modeling in order to align or register the 2-D, 3-D or 4-D ultrasound images within the patient or with respect to an external frame of reference. In this embodiment, the precise transducer array position and orientation information provided by the accelerometer sensors may be combined with X-ray or computer tomography (CT) scan data to register the ultrasound image data within the X, Y, Z coordinates of a patient-centered or external centered frame of reference. In this manner, structures detected in the ultrasound images (i.e., sources of ultrasound echoes) can be located at relatively precise points (e.g., at specific X, Y, Z coordinates) within the frame of reference.

Imaging the heart in 3-D and 4-D by making use of the various embodiments can be used to aid a physician in identifying locations for position a pacing lead on the heart in order to provide optimum benefit from a pacemaker. By generating accurate images of the surface of the heart throughout the cardiac cycle, the physician can locate regions that are not contracting in sequence or to the full extent as adjoining regions. By positioning pacing leads on such regions of the heart, a pacemaker may be better able to provide pacing stimulation to regions of the heart where such stimulation is most beneficial. Additionally, by imaging the heart in four dimensions, the physician can identify regions (such as portions of the left or right ventricle or atria) that are contracting early, late or otherwise out of phase with the rest of the heart. Lagging regions may be appropriate for placement of pacing leads. Additionally, the information on contraction lag contained in such a 4-D image dataset can be used to set the pace maker timing parameters. In this embodiment, the physician uses the 4-D image dataset (such as by running the 3-D images forward and backwards in time on the display) to calculate the time at which a lagging region of the heart should have contracted and uses this calculation to set the pacemaker timing parameter.

Additionally, 3-D and 4-D imaging according to various embodiments may be used to image the unsteady pacing area or malfunctioning area within the heart detected or located using ECG sensor data. U.S. Provisional Patent Application No. 60/795,912 entitled Method For Simultaneous Bi-Atrial Mapping Of Atrial Fibrillation, filed Apr. 29, 2006, which is incorporated herein by reference in its entirety, describes methods for locating malfunctioning areas of the heart using ECG data mapped on an anatomical model of the heart. By using the various embodiments to image the unsteady pace or otherwise malfunctioning region in the conductive pathway, the resulting images may enable the physician to more accurately locate and optimize the positions for pacing leads. Further, 4-D images of the selected region may enable the physician to more accurately optimize the pacing timing and rhythm for the lead, both by measuring the lag before emplacement to estimate an appropriate timing parameter and by measuring the lag after pacing is initiated to confirm the region is responding as desired to the pacing stimulation. Additionally, 3-D and 4-D images of the heart may be used to correct, correlate or otherwise improve the anatomical model used for displaying ECG data.

While the present invention has been disclosed with reference to certain exemplary embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

I claim:

1. An ultrasound imaging system, comprising:
a catheter,
an imaging transducer disposed near a distal end of the catheter;
a first accelerometer disposed proximate to the imaging transducer, the first accelerometer configured to sense translational acceleration of the imaging transducer; and
a computer configured to receive data from both the imaging transducer and the first accelerometer and adapted to integrate data from the imaging transducer and translational acceleration data from the first accelerometer to generate a multi-dimensional image.

2. The ultrasound imaging system as in claim 1, wherein the generated multi-dimensional image is a 3-D ultrasound image.

3. The ultrasound imaging system as in claim 1, wherein the computer is further adapted to calculate a translational displacement of the imaging transducer by calculating a second integral of translational acceleration data received from the first accelerometer.

4. The ultrasound imaging system as in claim 1, wherein the computer is further adapted to calculate a position of the imaging transducer based upon baseline position data and translational acceleration data received from the first accelerometer.

5. The ultrasound imaging system as in claim 4, wherein the computer is further adapted to calculate a position of the imaging transducer based upon baseline position data and the calculated translational displacement of the imaging transducer.

6. The ultrasound imaging system as in claim 1, further comprising a rotational sensor coupled to the catheter and configured to sense rotation of the catheter about an axis of rotation.

7. The ultrasound imaging system as in claim 1, further comprising a second accelerometer disposed proximate to the imaging transducer, the second accelerometer configured to sense rotational acceleration of the imaging transducer about an axis of rotation.

8. The ultrasound imaging system as in claim 6, wherein the computer is further adapted to calculate a rotational orientation of the imaging transducer based upon the sensed rotation of the catheter.

9. The ultrasound imaging system as in claim 7, wherein the computer is further adapted to calculate a rotational orientation of the imaging transducer based upon baseline rotational orientation data and rotational acceleration data received from the second accelerometer.

10. The ultrasound imaging system as in claim 1, further comprising:
a second accelerometer disposed proximate to the imaging transducer, the second accelerometer oriented on the catheter and configured to sense translational acceleration of the imaging transducer along an axis different from that of the first accelerometer; and
a third accelerometer disposed proximate to the imaging transducer, the third accelerometer oriented on the catheter and configured to sense translational acceleration of the imaging transducer along an axis different from that of the first and second accelerometers,
wherein the computer is further configured to receive data from the first and second accelerometers and adapted to integrate data from the imaging transducer and translational acceleration data from the first, second and third accelerometers to generate a multi-dimensional image.

11. The ultrasound imaging system as in claim 10, wherein the computer is further adapted to calculate a translational displacement vector of the imaging transducer by calculating a second integral of translational acceleration data received from the first, second and third accelerometers.

12. The ultrasound imaging system as in claim 10, wherein the computer is further adapted to calculate a position of the imaging transducer based upon baseline position data and translational acceleration data received from the first, second and third accelerometers.

13. The ultrasound imaging system as in claim 11, wherein the computer is further adapted to calculate a position of the imaging transducer based upon baseline position data and the calculated translational displacement vector of the imaging transducer.

14. The ultrasound imaging system as in claim 11, wherein the computer is further adapted to calculate a position of the imaging transducer as a vector addition of a baseline position vector and the calculated translational displacement vector of the imaging transducer.

15. The ultrasound imaging system as in claim 11, wherein:
at least one of the first, second and third accelerometers is configured to sense rotational acceleration, and
the computer is further adapted to calculate a rotational orientation of the imaging transducer based upon sensed rotational acceleration.

16. The ultrasound imaging system as in claim 13, wherein the generated multi-dimensional image is a 3-D ultrasound image.

17. The ultrasound imaging system as in claim 15, wherein the computer is further adapted to calculate a rotational displacement of the imaging transducer by calculating a second integral of sensed rotational acceleration data.

18. The ultrasound imaging system as in claim 16, wherein the computer is further adapted to calculate a position and orientation of image data from the imaging transducer based upon baseline position data, the calculated rotational displacement of the imaging transducer and acceleration data received from the first accelerometer.

19. The ultrasound imaging system as in claim 1, wherein baseline position data is provided by fluoroscopy.

20. The ultrasound imaging system as in claim 1, further comprising a magnet configured to apply a magnetic field to the first accelerometer so as to increase acceleration sensitivity of the first accelerometer.

21. The ultrasound imaging system as in claim 10, further comprising a magnet configured to apply a magnetic field to at least one of the first, second and third accelerometers so as to increase acceleration sensitivity of the first, second or third accelerometer.

22. An ultrasound imaging catheter, comprising:
an imaging transducer disposed near a distal end of the catheter; and
a first accelerometer disposed proximate to the imaging transducer, the first accelerometer configured to sense translational acceleration of the imaging transducer.

23. The ultrasound imaging catheter as in claim 22, further comprising a rotational sensor coupled to the catheter and configured to sense rotation of the catheter about an axis of rotation.

24. The ultrasound imaging catheter as in claim 22, further comprising a second accelerometer disposed proximate to the imaging transducer, the second accelerometer configured to sense rotational acceleration of the imaging transducer about an axis of rotation.

25. The ultrasound imaging catheter as in claim 22, further comprising:
a second accelerometer disposed proximate to the imaging transducer, the second accelerometer oriented on the catheter and configured to sense translational acceleration of the imaging transducer along an axis different from that of the first accelerometer; and
a third accelerometer disposed proximate to the imaging transducer, the third accelerometer oriented on the catheter and configured to sense translational acceleration of the imaging transducer along an axis different from that of the first and second accelerometers.

26. A method of displaying ultrasound images of an organ, the method comprising
positioning a distal end of the catheter within the organ with a first position and orientation, the catheter having an imaging transducer disposed near the distal end of the catheter and a accelerometer disposed proximate to the imaging transducer;
measuring a baseline position and orientation of the imaging transducer;
measuring an acceleration of the imaging transducer and calculating a displacement from the baseline position and orientation;
obtaining an image from the imaging transducer;
manipulating the catheter to a different orientation within the organ;
repeating the steps of measuring an acceleration and obtaining an image; and
generating a multi-dimensional image by combining the obtained images using the measured baseline position and measured accelerations.

27. The method of displaying ultrasound images of an organ of claim 26, wherein the organ is a heart and further comprising obtaining images throughout a cardiac cycle and generating multi-dimensional images of the heart throughout the cardiac cycle.

28. The method of displaying ultrasound images of an organ of claim 27, further comprising locating a position for attaching a pacemaker pacing lead based upon the multi-dimensional images the heart throughout the cardiac cycle.

29. The method of displaying ultrasound images of an organ of claim 27, further comprising setting a timing parameter for a pacemaker based upon the multi-dimensional images the heart throughout the cardiac cycle.

30. The method of displaying ultrasound images of an organ of claim 27, further comprising generating a magnetic field in the vicinity of the accelerometer so as to increase acceleration sensitivity of the accelerometer.
